(19) United States
(12) Patent Application Publication
(10) Pub. No.: US 2016/0222462 A1
(43) Pub. Date: Aug. 4, 2016

(54) Analysis of Apoptotic Bodies in Bodily Fluids

(71) Applicant: OncoMEDx, Inc., Hackettstown, NJ (US)

(72) Inventor: Michael S. Kopreski, Long Valley, NJ (US)

(21) Appl. No.: 15/004,813

(22) Filed: Jan. 22, 2016

Related U.S. Application Data
(63) Continuation of application No. 10/506,703, filed on Jun. 20, 2005, now abandoned.

(51) Int. Cl. C12Q 1/68; G01N 33/574

(52) U.S. Cl. CPC C12Q 1/6886; G01N 33/57419; G01N 2333/47; C12Q 2600/158

(57) ABSTRACT

The invention provides methods for the detection of apoptotic bodies in bodily fluid, including blood, blood plasma, and serum. The invention provides methods of labeling apoptotic bodies and their proteins, nucleic acids, and phospholipids. The apoptotic bodies may further be extracted, isolated, or separated from fractions of the bodily fluid.

### CROSS REFERENCE TO OTHER APPLICATIONS

application is a continuation of pending application Ser. No. 10/506,703 filed Jun. 20, 2005 which is a National Stage Entry of PCT/US03/07120 filed Mar. 10, 2003, which claims priority from Provisional Application 60/362,884 filed Mar. 8, 2002. These three applications are incorporated herein by reference in their entirety.

### BACKGROUND OF THE INVENTION

1. Field of the Invention

This invention relates to methods for the detection and analysis of products of apoptosis, particularly particles such as apoptotic bodies and apoptotic body fragments, in bodily fluids of a human or animal, whereby said detection and analysis enables the detection and characterization of the apoptotic body, thereby enabling the diagnosis, detection, evaluation, monitoring, or therapy of pathologic diseases characterized by apoptosis. The invention further provides methods for quantifying, separating, isolating, or concentrating apoptotic bodies and apoptotic body fragments from or within the bodily fluid of a human or animal, and further permits analysis of proteins and nucleic acids comprising the apoptotic body. Bodily fluids from which apoptotic bodies can be obtained according to the methods of this invention include blood, blood plasma, serum, urine, effusions (including pleural effusions, pericardial effusions, and joint effusions), ascites, saliva, cerebrospinal fluid, cervical secretions, amniotic fluid, gastrointestinal secretions (including secretions from the stomach, pancreas, liver, small intestine, and colon), sputum and bronchial secretions, breast fluid, synovial fluid, fluid removed from cysts, and tissue lavages. Apoptotic bodies are or comprise cellular fragments released, shed, or extruded from a cell during 20 apoptosis, wherein said fragments comprise cytoplasmic and/or nuclear remnants of the cell undergoing apoptosis, and thus comprise at least in part lipids, phospholipids, proteolipids, proteins, nucleoproteins, and/or nucleic acids including deoxyribonucleic acid (DNA) and ribonucleic acid (RNA). Such apoptotic bodies include cellular fragments of apoptosis of varying size, and any secondary fragmentation of apoptotic bodies.

2. Description of Related Art

Apoptosis is a common mechanism of cell death that occurs during many pathologic and normal processes. It is characterized morphologically by chromatin condensation, DNA fragmentation or degradation, mitochondrial membrane changes, cell fragmentation, and release of apoptotic bodies. The released or extruded apoptotic bodies are understood by the art to be thereafter phagocytized by local cells. It is thus understood by the art that apoptosis results in the orderly death and local disposal of a cell. However, it has not been demonstrated in this art that apoptotic bodies, including specifically apoptotic bodies derived from non-hematopoietic neoplastic cells, can escape local disposal and circulate in blood and blood plasma and serum, or are found in other bodily fluids.

DNA is known to circulate in plasma and serum (see U.S. Pat. Nos. 5,496,699; 5,952,170; 6,156,504; 6,521,409B1; Sorenson et al, Cancer Epidemiology, Biomarkers & Prevention, 1994, 3:67-71; Vasioukh. in et al, British Journal of Haematology, 1994, 86: 774-779; Chen et al., Clinical Cancer Research, 1999, 5: 2297-2303; Kopreski et al., Journal of the National Cancer Institute, 2000, 92: 918-923), said references incorporated herein in their entirety. Co-owned U.S. Pat. No. 6,329,179B1, incorporated herein by reference in its entirety, teaches detection of extracellular RNA in bodily fluids such as blood, blood plasma, and serum, and that such extracellular RNA can be amplified and detected from plasma and serum. In particular, tumor-associated RNA has been demonstrated in plasma and serum (Kopreski. et al., Clinical Cancer Research, 1999, 5: 1961-1965; Chen et al, Clinical Cancer Research. 2000, 6: 3823-3826; Hasselmann et al., Oncology Reports, 2001, 8: 115-118; Dasi et al., Laboratory Investigation, 2001. 81: 767-769; said references incorporated herein in their entirety). At least a portion of this RNA may be present in plasma or serum as components of apoptotic bodies. Furthermore, in vitro evidence suggests tumor-associated RNA within apoptotic bodies is protected from nucleases present in blood (Hasselmann et al., Clinical Chemistry, 2001, 47: 1488-1489, incorporated herein in its entirety).

Thus, there is a need in the art to detect apoptotic bodies associated with normal cell death as well as tumor cell and other pathologic instances of cell death, in order to detect the existence and extent of apoptosis as a measure of health or disease in an animal, including a human.

### SUMMARY OF THE INVENTION

The present invention teaches methods of detecting and analyzing apoptotic bodies and their components, including in particular nucleic acids and proteins including nuclear matrix proteins and ribonucleoproteins, and uses and applications thereof. The invention is particularly useful for diagnosis, detection, evaluation, monitoring, prognosticating, or therapeutically assessing pathologic diseases characterized by apoptosis, including but not limited to cancer and premalignancy.

The present invention provides methods for detection and analysis of apoptotic bodies in blood, plasma, serum, and other bodily fluids from an animal, most preferably a human. The invention thereby provides a method for the diagnosis, detection, evaluation, monitoring, prognostication, or therapy of pathologic diseases associated with apoptosis, said diseases including in particular neoplastic diseases such as cancer and premalignant diseases, and non-neoplastic diseases such as cardiovascular diseases and neurologic or neurodegenerative diseases. The invention provides methods for isolating or identifying the apoptotic body in the bodily fluid, and provides thereby for the analysis of the proteins and nucleic acids of the apoptotic body. Said analysis of protein, DNA, and/or RNA may be performed separately, sequentially, or concomitantly.

In a preferred embodiment, the invention provides methods for extracting, isolating, or concentrating apoptotic bodies in a bodily fluid of a human or animal, the method comprising the steps of contacting the bodily fluid with a primer or probe specific for a protein, phospholipid, or nucleic acid present in an apoptotic body, conjugating or hybridizing the primer or probe to the protein, phospholipid, or nucleic acid, and thence separating the primer or probe from the bodily fluid to extract, isolate, or concentrate apoptotic bodies. In a preferred embodiment of the invention, the bodily fluid is blood, blood plasma or serum. urine, effusions (including pleural effusions, pericardial effusions, and joint effusions), ascites, saliva, cerebrospinal fluid, cervical secretions, amniotic fluid, gastrointestinal secretions (including secretions from the stomach, pancreas, liver, small intestine, and colon), sputum and bronchial secretions, breast fluid, synovial fluid, fluid removed from cysts, or tissue lavages. In one preferred embodiment the bodily fluid is a non-cellular fraction of a blood, urine, effusions (including pleural effusions, pericardial effusions, and joint effusions), ascites, saliva, cerebrospinal fluid, cervical secretions, amniotic fluid, gastrointestinal secretions, sputum and bronchial secretions, breast fluid, synovial fluid, fluid from a cyst, or tissue lavages. In a particularly preferred embodiment the bodily fluid is plasma or serum.

The invention provides methods for labeling and detecting or otherwise identifying a protein component of the apoptotic body, whereby the protein is detected in the bodily fluid, the method comprising the steps of labeling the protein with a probe or antibody or labeling moiety that binds specifically to a protein present in the apoptotic body, or specifically to a class of epitopes or moieties comprising the protein, and detecting the label. In one aspect of this embodiment, the apoptotic body is extracted or purified from the bodily fluid prior to labeling and detecting the protein. In another aspect of this embodiment, the labeling moiety is an antibody such as a monoclonal antibody, wherein the antibody labels or detects a protein component of the apoptotic body. In particularly preferred embodiments, the protein is a nuclear matrix protein or a ribonucleoprotein. In a particularly preferred embodiment, the labeling moiety is annexin V.

The invention further provides methods for labeling a phospholipid component of the apoptotic body, the method comprising the step of labeling the phospholipid with a labeling probe or moiety that binds to the phospholipid, and detecting the label. In preferred embodiments of the inventive methods, the labeling probe or antibody or is conjugated with a fluorescent moiety, a radioisotope, biotin, or a chromophore that enables detection of the label, whereby the label is detected. In particularly preferred embodiments, the labeled probe or moiety is detected by gel electrophoresis, capillary electrophoresis, enzyme-linked immunosorbent assay (ELISA) or modifications thereof, such as using biotinylated or otherwise modified primers, fluorescent-, radioisotope- or chromogenically-labeled probe, laser-induced fluorescence detection, Western blot analysis, Northern blot analysis, Southern blot analysis, electrochemiluminescence, reverse dot blot detection, high-performance chromatography, spectroscopy including mass spectroscopy and nuclear magnetic resonance spectroscopy, flow cytometry, laser scanning cytometry, or detection at bioelectrical interfaces.

In one aspect of the invention, the labeling probe or antibody is attached to a solid substrate capable of binding apoptotic bodies or components thereof In one embodiment, the probe or antibody attached to a solid substrate facilitates extraction, purification, separation, or isolation of apoptotic bodies from the bodily fluid. In one aspect of this embodiment, apoptotic bodies are thereby concentrated from the bodily fluid. In another aspect of this embodiment, proteins and nucleic acids associated with apoptotic bodies are thereby extracted, purified, separated, isolated, or concentrated from the bodily fluid.

The invention thereby provides kits comprising one or a plurality of probes, primers, or antibodies attached to a solid substrate that permit extraction, purification, separation, isolation, or concentration of apoptotic bodies, and thereby their associated proteins and nucleic acids, from bodily fluids. In certain embodiments, said kits provide said substrate as a solid surface upon which the bodily fluid contacts or passes over, such as a column, filter, or solid layer. In other embodiments, said kits provide said substrate that is capable of being mixed with the bodily fluid. In certain embodiments, the solid substrate is a solid bead or particle, for example a magnetic bead or particle.

The invention further provides kits comprising one or a plurality of filters having a pore size that is less than the size of a cell, preferably less than 6 microns and most preferably less than 1 micron, but preferably greater than 0.1 micron, for extracting, purifying, separating, or concentrating apoptotic bodies from a bodily fluid.

In other aspects of the invention, labeling probes or antibody moieties bind to a protein or nucleic acid that is a disease-specific or disease-associated indicator. In a particularly preferred embodiment, the disease is a neoplastic disease such as cancer or premalignancy. In other preferred embodiments, the disease is a non-neoplastic disease, including but not limited to a cardiovascular disease or a neurologic disease.

The invention also provides methods for labeling nucleic acid components of the apoptotic body, these methods comprising the steps of hybridizing an oligonucleotide primer or a probe specific for a nucleic acid of the apoptotic body, or to cDNA derived therefrom. In one aspect of this embodiment, the nucleic acid is ribonucleic acid (RNA). In another aspect of this embodiment, the nucleic acid is deoxyribonucleic acid (DNA). In another aspect of this embodiment, the nucleic acid is a component of a ribonucleoprotein. In another aspect of this embodiment, the nucleic acid is first extracted from the apoptotic body prior to hybridization. In another aspect of this embodiment, the nucleic acid is hybridized within the apoptotic body.

In certain embodiments, the invention provides such methods whereby the hybridized nucleic acid of the apoptotic body, or cDNA. derived therefrom, is amplified in a qualitative or a quantitative manner prior to detection. In one aspect of these embodiments, nucleic acids are amplified using a method that is polymerase chain reaction, reverse transcription polymerase chain reaction, ligase chain reaction, DNA signal amplification, amplifiable RNA reporters, Q-beta replication, transcription-based amplification, isothermal nucleic acid sequence based amplification, self-sustained sequence replication assays, boomerang DNA amplification, strand displacement activation, cycling probe technology, or any combination or variation thereof. In other aspects of this embodiment, the amplified products are detected using a methods that is gel electrophoresis, capillary electrophoresis, conventional enzyme-linked immunosorbent assay (ELISA) or modifications thereof, such as amplification using biotinylated or otherwise modified primers, nucleic acid hybridization using specific, detectably-labeled probes, such as fluorescent-, radioisotope-, or chromogenically-labeled probe, laser-induced fluorescence detection, Northern blot analysis, Southern blot analysis, electrochemiluminescence, reverse dot blot detection, high-performance liquid chromatography, spectroscopy, or detection at bioelectrical interfaces.

The invention provides methods for extracting, isolating, separating, or concentrating apoptotic bodies from a bodily fluid to enhance detection and analysis of the apoptotic bodies, the method comprising the steps of obtaining a bodily fluid and extracting, isolating, separating, or concentrating apoptotic bodies in the bodily fluid from the cellular fraction of the bodily fluid. In a particularly preferred embodiment, apoptotic bodies are extracted from the bodily fluid. In particularly preferred embodiments of the invention, the separated or extracted apoptotic bodies are labeled apoptotic bodies. In a particularly preferred embodiment of the invention, the bodily fluid is plasma or serum. In one aspect of these embodiments, apoptotic bodies in a bodily fluid are separated or extracted using a capturing probe attached to a magnetic bead or particle, such as an iron or steel particle, thereby facilitating separation or extraction performed within a magnetic field, whereby apoptotic body-metal particle conjugates are drawn in the direction of the magnetic field. In another aspect of this embodiment, apoptotic bodies in a bodily fluid are separated or extracted using a capturing probe attached to a solid substrate, thereby facilitating separation or extraction of the apoptotic bodies.

In other aspects of this embodiment, apoptotic bodies are separated from the cellular fraction of a bodily fluid by centrifugation, wherein the non-cellular centrifuged fraction contains the apoptotic body. In other aspects of this embodiment, apoptotic bodies are separated from the cellular fraction of a bodily fluid by passing the bodily fluid through a filter, wherein the apoptotic bodies pass through the filter and the cellular fraction of the bodily fluid does not pass through the filter, thereby separating the fraction of the bodily fluid containing the apoptotic body. In a preferred embodiment, the pores of the filter are smaller than 1 micron. In a particularly preferred embodiment, the pores of the filter are smaller than 0.5 microns.

In another preferred embodiment of the invention, apoptotic bodies are extracted, isolated, purified, or concentrated from a bodily fluid to enhance detection and analysis of the apoptotic body, the method comprising the steps of obtaining a bodily fluid and extracting, isolating, purifying, or concentrating apoptotic bodies from the bodily fluid. In a particularly preferred embodiment of the invention, the bodily fluid is plasma or serum. In one aspect of this embodiment, apoptotic bodies in a bodily fluid are extracted, isolated, purified, or concentrated from a bodily fluid using a capturing probe attached to a magnetic bead or particle, such as an iron or steel particle, thereby facilitating extraction, isolation, purification, or concentration of the bodily fluid, wherein the method is performed within a magnetic field, and wherein the apoptotic body-metal particle conjugate is drawn in the direction of the magnetic field. In another aspect of this embodiment, apoptotic bodies are extracted, isolated, purified, or concentrated from a bodily fluid using a capturing probe attached to a solid substrate placed into contact with the bodily fluid, and thereby facilitating extraction, isolation, purification, or concentration of apoptotic bodies from the bodily fluid.

In another aspect of this embodiment, apoptotic bodies are extracted, isolated, purified, or concentrated from a bodily fluid following centrifugation and removal of the bodily fluid from centrifuged apoptotic bodies.

In another aspect of this embodiment, apoptotic bodies are extracted, isolated, purified, or concentrated following a step of evaporating or desiccating the bodily fluid.

In preferred embodiments of the invention, the capture probe is an antibody, for example a monoclonal antibody, or an oligonucleotide. In this aspect the capturing probe may be an antisense oligonucleotide.

In another preferred embodiment of the invention, apoptotic bodies present in a bodily fluid are labeled by contacting the bodily fluid with a labeling probe or primer moiety specific for a phospholipid, protein, or nucleic acid present in the apoptotic body. In an aspect of this embodiment, labeled apoptotic bodies are thereafter detected or analyzed directly in the bodily fluid, such as by flow cytometry or spectrometry.

In another preferred embodiment of the invention, apoptotic bodies in a bodily fluid are passed through an electrical gradient, whereby apoptotic bodies are isolated or separated from other components of the bodily fluid on the basis of electric charge.

In one aspect of this embodiment, isolated or extracted, isolated, purified, concentrated, or separated apoptotic bodies are analyzed to identify a protein or nucleic acid component thereof. In particularly preferred embodiments, the protein identified is a nuclear matrix protein or a ribonucleoprotein. In particularly preferred embodiments, the nucleic acid identified is RNA or DNA. In particularly preferred embodiments, the apoptotic body protein or nucleic acid is analyzed or identified by comparing a physical profile obtained by nucleic acid amplification, spectroscopy (including mass spectroscopy or nuclear magnetic spectroscopy), flow cytometry, signal amplification, or by laser-induced fluorescence to a known protein or nucleic acid physical profile.

In one aspect of the invention, the apoptotic body is analyzed at a bioelectric interface.

The methods of the invention are advantageously used for providing a diagnosis or prognosis of, for monitoring, or as a predictive indicator for neoplastic and non-neoplastic diseases or pathologic conditions and injuries. The methods of the invention are particularly useful for monitoring or providing a diagnosis or prognosis of cancer and premalignancy. The methods of the invention further provide ways to identify animals, most preferably humans, having non-neoplastic disease or pathologic conditions. The methods of the invention thereby permit rational, informed treatment options to be used for making therapeutic decisions.

Other advantageous uses for the methods of the invention include providing a marker for assessing or monitoring the adequacy or efficacy of a therapy, or for determining whether additional or more advanced therapy is required. The invention therefore provides methods for developing a prognosis in such patients.

Other advantageous uses for the methods of the invention for screening individuals to determine their predisposition for a disease or pathologic condition, and further to determine their need for additional diagnostic evaluation and/or for preventive therapy.

Specific preferred embodiments of the present invention will become evident from the following more detailed description of certain preferred embodiments and the claims.

### DETAILED DESCRIPTION OF THE INVENTION

The invention provides methods for detecting and analyzing apoptotic bodies in blood, blood plasma, serum, and other bodily fluids such as urine, effusions (including pleural effusions, pericardial effusions, and joint effusions), ascites, saliva, cerebrospinal fluid, cervical secretions, amniotic fluid, gastrointestinal secretions, sputum and bronchial secretions, breast fluid, synovial fluid, fluid from cysts, and tissue lavages, from an animal, most preferably a human. The invention provides methods for diagnosing, detecting, evaluating, monitoring, or providing a prognosis or therapy for pathologic diseases associated with apoptosis.

Pathologic diseases associated with apoptosis include but not to be limited to cancer and premalignant conditions, cardiovascular diseases, neurologic diseases, and diseases of other organ systems. Premalignant conditions or diseases include but are not limited to adenoma such as colorectal adenoma, dysplasia, prostatic intraepithelial neoplasia (PIN), cervical dysplasia, cervical intraepithelial neoplasia (CIN), bronchial dysplasia and metaplasia, atypical hyperplasia of the breast, ductal carcinoma-in-situ, atypical endometrial hyperplasia, and Barrett's esophagus.

The methods of the invention are particularly advantageous for detecting and monitoring non-hematopoietic cancer and premalignancy. As used herein, non-hematopoietic cancers or malignancy include but are not limited to breast cancer, prostate cancer, ovarian cancer, lung cancer, cervical cancer, colorectal cancer, gastric cancer, hepatocellular cancer, pancreatic cancer, gallbladder cancer, bladder cancer, renal cancer, melanoma, esophageal cancer, head and neck cancer, sarcomas, and cancers of the brain.

The invention further enables detection and monitoring of hematopoietic cancers or malignancy, wherein hematopoietic cancers or malignancy include but are not limited to lymphoma, multiple myeloma, and leukemia (such as acute myelogenous leukemia, acute lymphocytic leukemia, chronic lymphocytic leukemia, and chronic myelogenous leukemia).

The methods of the invention comprise the steps of obtaining a bodily fluid, thereafter conjugating, hybridizing, or labeling a protein or nucleic acid component of apoptotic bodies in said bodily fluid with a primer, probe, or antibody or other labeling moiety, and thereafter detecting or analyzing the labeled-apoptotic bodies or proteins or nucleic acids thereof. In preferred embodiments, the bodily fluid may be a non-cellular fraction of blood, urine, effusions, ascites, saliva, cerebrospinal fluid, cervical secretions, amniotic fluid, gastrointestinal fluid or secretions, sputum and bronchial secretions, breast fluid, synovial fluid, fluid from a cyst, or tissue lavage. It is particularly preferred that the non-cellular fraction is plasma or serum. The labeled product may be detected or analyzed directly within the bodily fluid, or it may be separated, isolated, purified, or extracted from the bodily fluid, or concentrated within or apart from the bodily fluid prior to analysis. In the practice of the methods of the invention, proteins or nucleic acids may be but need not be amplified or signal amplified in a qualitative or quantitative fashion either prior to or following labeling.

In the practice of the methods of the invention, apoptotic bodies may be extracted, purified, isolated, concentrated, separated, or labeled from any bodily fluid, including but not limited to whole blood, plasma, serum, urine, effusions, ascitic fluid, saliva, cerebrospinal fluid, cervical secretions, vaginal secretions, endometrial secretions, gastrointestinal fluids and secretions including fluids and secretions from the stomach, pancreas, liver, gallbladder, small intestines, and colon, bronchial secretions including sputum, breast fluid or secretions, or washings or lavages. In a preferred embodiment, the bodily fluid is either blood plasma or serum. The blood may be drawn by routine venipuncture, or by finger-stick or capillary stick or from an indwelling venous access device such as a venous catheter. In a preferred embodiment, the blood is drawn by venipuncture with from 1-10 milliliters of blood obtained, although lesser amounts or greater amounts are acceptable. It is preferred, but not required, that the blood be processed or frozen soon after drawing, and preferably within 6 hours if apoptotic body-derived nucleic acid is to be hybridized or labeled, and within 24 hours if apoptotic body-derived protein is to be conjugated or labeled. In a preferred embodiment, blood is first collected by venipuncture and kept on ice until use or processing. Preferably, within 1-3 hours of drawing the blood, plasma or serum is separated from the cellular fraction by centrifugation of blood, for example at 1100×g for 10 minutes at 4 degrees C. When using plasma, the blood is not permitted to coagulate prior to separation of the cellular and acellular components. Serum or plasma can be frozen if storage is desired, most preferably at −20 degrees C. to −80 degrees C. after separation from the cellular portion of blood until further assay. When the frozen specimen is thawed for further assay, it should be thawed rapidly if apoptotic body nucleic acids are being assayed, for example in a 37 degrees C. water bath. Repetitive freeze-thawing of the specimen should be avoided as degradation of the apoptotic body may occur following each freeze-thaw cycle. In the preferred embodiments, the number of freeze-thaw cycles should not exceed 2, and is preferably limited to a single cycle.

In the practice of the methods of the invention, protein, nucleic acid, or phospholipid components of the apoptotic bodies in the bodily fluid are conjugated, hybridized, or labeled, whereby said protein, nucleic acid, or phospholipid is thereby detected and analyzed. Apoptotic body components are in certain embodiments labeled directly within the bodily fluid, or said components in other embodiments are extracted, isolated, separated, purified, or concentrated from the bodily fluid either prior to conjugation or labeling or following conjugation or labeling. Intact apoptotic bodies are labeled when protein, phospholipid, or nucleic acid species of interest are present on the surface membrane of the apoptotic body. In addition, labeling or extracting other proteins or nucleic acids of interest is facilitated by disrupting apoptotic bodies, for example, by mechanical, ultrasound, microwave, or chemical methods such as lysis buffers and phospholipid solvents.

The invention provides methods for isolating or separating apoptotic bodies from the cellular fraction of the bodily fluid to enhance detection and analysis of apoptotic bodies or protein or nucleic acid components thereof. In preferred embodiments, the bodily fluid may be a non-cellular fraction of blood, urine, effusions, ascites, saliva, cerebrospinal fluid, cervical secretions, amniotic fluid, gastrointestinal fluid or secretions, sputum and bronchial secretions, breast fluid, synovial fluid, fluid from a cyst, or tissue lavage. It is particularly preferred that the non-cellular fraction is plasma or serum. The use of these methods of the invention permits apoptotic bodies within cellular bodily fluids to be analyzed, inter alia, for specific protein, nucleic acid or lipid content. In a preferred embodiment, separation of apoptotic bodies from the cellular fraction of bodily fluids is performed by centrifugation of the bodily fluid to separate the cellular fraction of the bodily fluid from that fraction of the bodily fluid containing apoptotic bodies. In one preferred method, whole blood is centrifuged at greater than 500×g, and preferably 800-1200×g, whereby the cellular component of blood is separated from the plasma or serum fraction, and wherein apoptotic bodies remain within the plasma or serum fraction. Upon fractionating apoptotic bodies from the cellular fraction of blood, if desired apoptotic bodies may be further separated or isolated from the plasma or serum fraction by centrifuging again at higher speeds, upon which the apoptotic bodies will centrifuge from the fluid fraction.

In other preferred embodiments, apoptotic bodies are separated from the cellular fraction of a bodily fluid bypassing the bodily fluid through a filter, wherein the apoptotic body passes through the filter, and the cellular fraction of the bodily fluid does not pass through the filter, thereby separating the cellular fraction and the apoptotic body fraction of the bodily fluid. In a preferred embodiment, the pores of the filter are smaller than cell size, preferably less than 6 microns and most preferably less than 1 micron. In particularly preferred embodiments, filter pores are smaller than 0.5 microns. For example, but not a limitation, the filter may be a 0.45 micron cellulose acetate filter (Nalgene, Rochester N.Y.).

In other preferred embodiments, the bodily fluid is passed through multiple filters, wherein the first filter separates apoptotic bodies from the cellular fraction of the bodily fluid, and wherein the subsequent filter(s) separates or isolates apoptotic bodies from the remaining bodily fluid, thereby providing an isolated fraction comprising to large degree the apoptotic body fraction. This isolated fraction, which can be detectably labeled prior to fractionation or subsequent to fractionation, may then be further analyzed for specific protein, nucleic acid, or phospholipids components.

The invention also provides kits comprising in whole or part of multiple filters comprised of pores or differing size that enables separation of the apoptotic body fraction according to the methods described herein.

Apoptotic bodies can also be isolated or separated from the cellular fraction of a bodily fluid by both centrifuging and filtering the bodily fluid in a combined fashion, for example by first centrifuging whole blood to provide plasma or serum, and thereafter filtering the plasma or serum as described.

In preferred embodiments of the invention, a bodily fluid comprising apoptotic bodies is passed over a solid substrate, where upon the apoptotic bodies attach to the solid substrate, thereby facilitating their extraction, purification, concentration, isolation, or separation from the bodily fluid. In preferred embodiments, the bodily fluid may be a non-cellular fraction of blood, urine, effusions, ascites, saliva, cerebrospinal fluid, cervical secretions, amniotic fluid, gastrointestinal fluid or secretions, sputum and bronchial secretions, breast fluid, synovial fluid, fluid from a cyst, or tissue lavage. It is particularly preferred that the non-cellular fraction is plasma or serum. The solid substrate can be conjugated to capturing primers, probes or labels specific to proteins or nucleic acids or phospholipids of the apoptotic bodies, whereby the apoptotic bodies become attached to the solid substrate. The solid substrate can thus be placed in contact with the bodily fluid, or the bodily fluid made to pass over the solid, to promote contact between the primers or probes and the apoptotic bodies. The solid substrate can thereby provide a bioelectric interface for detecting specific proteins or nucleic acids associated with the apoptotic body, or may provide a chip or surface for further amplification or detection as known in the art. In certain embodiments, the bodily fluid is first centrifuged and/or filtered prior to being passed over the solid substrate, or the bodily fluid washed directly over the solid substrate.

The invention further provides kits comprising in whole or in part said solid substrate for isolating or separating apoptotic bodies from a bodily fluid. Further, the invention provides within said kits solid substrate that are directly used for detecting or analyzing apoptotic bodies, or for proteins, nucleic acids, or phospholipid components of apoptotic bodies. Kits of the invention further incorporate specific oligonucleotide primers or antibody probes such as monoclonal antibodies, oligonucleotides, or antisense oligonucleotides attached to the solid as capturing probes. Said solid substrate in certain embodiments is provided as a filter, column, or solid layer, or provided as a substrate to be mixed within the bodily fluid, such as a solid bead or particle, such as a magnetic bead or particle. The kits of the invention can further provide reagents for amplification, reagents for nucleic acid extraction, and components for detection. The inventive kits can further include standard controls or controls to facilitate quantitative amplification. The kits can also further include a bioelectric interface.

In a preferred embodiment, apoptotic bodies in a bodily fluid are separated or extracted from the bodily fluid using a label, primer, or probe attached to a magnetic bead, iron, or steel particle, thereby facilitating separation or extraction of apoptotic bodies, wherein the apoptotic bodies bind to the magnetic beads or particles. The capturing probe may be, but is not limited to a primer or probe that hybridizes to a nucleic acid, or an antibody that is specific to a protein, comprising the apoptotic bodies. Probes can also be conjugated to a detector component that is a fluorescent moiety, radioisotope, or chromogenic label allowing detection of the protein or nucleic acid. The capturing probe can be an oligonucleotide, including an antisense oligonucleotide, or a monoclonal antibody. The primers or probes are also advantageously provided unlabeled in said kits of the invention, for later labeling by a user. Alternatively, such unlabeled probes or primers can be used unlabeled in methods for separating or extracting apoptotic bodies from a bodily fluid as disclosed herein. Apoptotic bodies separated or extracted using said unlabeled primers of probes of the kits of the invention can be further analyzed by amplification, spectroscopy, or immunologic or biochemical evaluation. In preferred embodiments, apoptotic bodies are bound to magnetic beads or particles drawn under the direction of a magnetic field and separated, extracted and isolated thereby. Preferably, the labeled magnetic beads or particles are specific for a nucleic acid or protein component of interest comprising the apoptotic body.

In alternative preferred embodiments of the invention, apoptotic bodies in a bodily fluid are separated or analyzed and differentiated by passing the bodily fluid through an electrical gradient, whereby the apoptotic body is isolated or separated from other components of the bodily fluid on the basis of electric charge.

In the practice of the methods of certain aspects of the invention, the separated, extracted, isolated, identified or labeled apoptotic bodies are analyzed to identify one or a plurality of specific proteins or nucleic acids that comprise the apoptotic body. In particularly preferred embodiments, the protein identified is a nuclear matrix protein or a ribonucleoprotein, including but not limited to telomerase or telomerase associated nucleic acid or protein (e.g., hTR, hTERT, or TEP1). In particularly preferred embodiments, the nucleic acid analyzed is a DNA or RNA. In the practice of the methods of these aspects of the invention, RNA can first be reverse transcribed to its corresponding cDNA and thereafter amplified or analyzed. Nucleic acid species are extracted from apoptotic bodies and amplified in a qualitative or quantitative manner. In preferred embodiments, methods of extracting and amplifying nucleic acid species useful in the practice of this invention are those disclosed in co-owned U.S. Pat. No. 6,329,179B1, incorporated herein by reference in its entirety. Methods of extracting nucleic acid from apoptotic bodies include but are not limited to gelatin extraction, silica, glass bead, or diatom extraction, guanidine-thiocyanate-phenol solution extraction, guanidinium-thiocyanate acid-based extraction, centrifugation through a cesium chloride or similar gradient, salt-based extraction methods, and phenol-chloroform-based extraction methods. Methods of amplifying nucleic acids extracted from apoptotic bodies include but are not limited to polymerase chain reaction, reverse transcription polymerase chain reaction, ligase chain reaction, branched DNA signal amplification, amplifiable RNA reporters, Q-beta replication, transcription-based amplification, isothermal nucleic acid sequence based amplification, self-sustained sequence replication assays, boomerang DNA amplification, strand displacement activation, cycling probe technology, and/or modifications thereof. It will be understood by those with skill in the art that other methods of nucleic acid amplification, including other methods of signal amplification, as well known in the art, may be applied.

The methods of the invention are provided so that amplified products are thereafter detected, for example, but not a limitation, by gel electrophoresis, capillary electrophoresis, conventional enzyme-linked immunosorbent assay (ELISA) or modifications thereof, such as amplification using biotinylated or otherwise modified primers, nucleic acid hybridization using specific, detectably-labeled probes, such as fluorescent-, radioisotope-, or chromogenically-labeled probe, laser-induced fluorescence detection, Northern blot analysis, Southern blot analysis, electrochemiluminescence, reverse dot blot detection, high-performance liquid chromatography, spectroscopy, or detection at bioelectrical interfaces.

In other preferred embodiments, apoptotic bodies protein or nucleic acid components thereof are analyzed by spectroscopy, including but not limited to mass spectroscopy and nuclear magnetic resonance spectroscopy. In other preferred embodiments, apoptotic bodies are analyzed by flow cytometry. In other preferred embodiments, apoptotic bodies are detected and analyzed by signal amplification or by laser-induced fluorescence.

In certain embodiments of the invention, apoptotic bodies are analyzed at a bioelectric interface. The bioelectric interface can be a solid substrate, wherein a primer or probe such as a capture oligonucleotide strand or capture monoclonal antibody is adhered to the solid substrate. Preferably, said capture monoclonal antibody or oligonucleotide combines specifically with a protein or nucleic acid component, respectively, of the apoptotic body, wherein the conjunction of the protein or nucleic acid of the apoptotic body from bodily fluid with the capture primer or probe thereby directly or indirectly generates an electrical signal. Said electric signal may further be amplified or similarly detected, inter alfa, using capacitance or conductivity measurements, for example using the method of Park et al. (Science 2002, 295: 1503-1505}, incorporated herein by reference in its entirety. The invention provides methods whereby the bioelectrical interface occurs directly with the apoptotic body, or alternatively whereby the protein or nucleic acid species of interest is first extracted from the apoptotic body. In the latter embodiments, protein and nucleic acid components are preferably extracted using the extraction methods disclose herein above, or alternatively by microwave extraction, wherein thereafter the protein or nucleic acid component interfaces with the bioelectric solid substrate. In certain embodiments, the protein or nucleic acid species interfaces with the bioelectric solid within a solution or suspension such as the bodily fluid itself or a salt solution or one of the extraction solutions. Said solution may further include a nuclease inhibitor to facilitate stability in the extracted nucleic acid. In one aspect of these embodiments, RNA extracted from the apoptotic body is first reverse transcribed to cDNA prior to exposure to the bioelectrical interface. In other aspects of these embodiments, RNA extracted from the apoptotic body is directly captured to generate an electrical signal. In other aspects of these embodiments, DNA extracted from apoptotic bodies is directly captured to generate an electrical signal. It will be understood that multiple DNA and RNA species may be concurrently or sequentially so captured using these methods.

In certain aspects of the methods of the invention, one or a plurality of specific proteins from apoptotic bodies combine into a specific antibody conjugate, wherein the antibody conjugate thereafter specifically combines with a capture probe on a solid substrate, including but not limited to a capture probe at a bioelectric interface.

The methods of the invention are also provided whereby the steps for extracting, purifying, concentrating, separating or isolating apoptotic bodies in bodily fluid are combined with steps for amplifying or signal amplifying or detecting protein or nucleic acid components of said apoptotic bodies, most preferably using the kits of the invention. In preferred embodiments, the inventive kits are adapted by the primers, probes, reagents or instructions comprising said kits to be diagnostic kits useful in detecting protein or nucleic acid components of apoptotic bodies. In a particularly preferred embodiment of the invention, diagnostic kits comprise a filter enabling separation of apoptotic bodies from the cellular fraction of a bodily fluid, and a bioelectric interface enabling detection of protein or nucleic acid components of apoptotic bodies. The methods of the invention are also provided wherein the steps for extracting, purifying, concentrating, separating or isolating apoptotic bodies in bodily fluid are combined with steps for amplifying or signal amplifying or detecting protein or nucleic acid components of said apoptotic bodies, in a sequential manner. For example, but not by way of limitation, in sequence the bodily fluid is centrifuged to separate the cellular fraction of the fluid from the apoptotic body fraction of the fluid, where upon protein or nucleic acid components are thereafter detected using a bioelectrical interface.

The methods of the invention permit multiple proteins and/or nucleic acids components of apoptotic bodies to be detected, captured, or amplifies sequentially or in combination as described herein. The invention therefore provides for a multiplex assay approach, advantageously using an array, microarray or microchip approach, including but not limited to bioelectrical interface drips. All methods for detecting or amplifying as described herein may be performed in either qualitative or quantitative fashion. Furthermore, qualitative or quantitative results detecting the presence or absence of specific proteins or nucleic acids in the apoptotic bodies may be compared to populations of individuals with specific disease and without specific disease, as to assess probability of the subject having disease. Furthermore, said probability is advantageously assessed by comparing multiple data points comprising the presence or absence of multiple apoptotic body protein and/or nucleic acid markers within a mathematical model whereby disease is thereby predicted.

In particularly preferred embodiments of the invention, the nucleic acids of interest derived or obtained from apoptotic bodies are cancer-associated DNA and cancer-associated RNA. Cancer-associated DNA includes DNA from both mutated and non-mutated genes, including but not limited to K-ras, H-ras, N-ras, c-myc, her-2/neu, bcr-abl, fms, src, fos, sis, jun, bcl-2, bc1-2/IgH, Von Hippel-Lindau gene, P53, retinoblastoma gene, mutated in colon cancer gene (MCC), deleted in colon cancer gene (DCC), epidermal growth factor gene, epidermal growth factor receptor gene, multi-drug resistance genes, microsatellite DNA alterations, p16, the Wilm's tumor gene WT1, hypermethylated DNA, and other oncogenes and tumor-suppressor genes. Cancer-associated RNA includes RNA from the cancer-associated DNA, including but not limited to tyrosinase RNA, cytokeratin RNA, prostate specific antigen RNA, alpha-fetoprotein RNA, carcinoembryonic antigen RNA, p97 RNA, MUC 18 RNA, PML/RAR RNA, CD44 RNA, EWS/FLI-1 RNA, EWS/ERG RNA, AML1/ETO RNA, MAGE RNA, beta human chorionic gonadotropin RNA, 5T4 RNA, COX-2 RNA, telomerase RNA, including telomerase RNA template (hTR) RNA, and telomerase reverse transcriptase protein (hTERT) RNA. Cancer-associated nucleic acids may be mutated, translocated, hypermethylated, or may otherwise demonstrate a DNA alteration and other epigenetic alteration.

The invention further provides methods for labeling apoptotic bodies with detectably-labeled probes. In preferred methods of the invention, one or a plurality of protein species comprising apoptotic bodies are labeled using a probe or antibody that specifically binds to the protein, or to epitopes or moieties comprising the protein. In particularly preferred embodiments, the antibody is a monoclonal antibody specific to a particular protein, including such non-limiting examples as nuclear matrix proteins or ribonucleoproteins. In particularly preferred embodiments, protein is a ribonucleoprotein that is telomerase. In other particularly preferred embodiments, the protein is a ribonucleoprotein that is heterogeneous nuclear ribonucleoprotein (hn RNP), including but not limited to hn RNP A1, hn RNP A2/B 1, hn RNP B 1, and hn RNP K. In preferred embodiments, the labeling moiety is annexin V, which binds to phosphatidylserine present on the apoptotic body membrane. Annexin V can be labeled with a radioisotope to identify it, or with a fluorescent or chromogenic label Further, annexin V can be attached to a solid substrate or to a magnetic or metallic bead or particle. Annexin V, and preferably annexin V attached to a solid substrate or to a magnetic or metallic bead or particle can be mixed in solution with apoptotic bodies, or can be washed over apoptotic bodies already isolated or attached to solid substrates. Apoptotic bodies isolated using these embodiments of the inventive methods can thereby be detected immunochemically. Apoptotic bodies can further be detected by double staining, for example using annexin V-propidium iodide double staining.

The invention further permits any combination of labeling and extraction or isolation and detection of the apoptotic body in bodily fluid. For example, but not a limitation, apoptotic bodies can be extracted, isolated, or concentrated from bodily fluid onto a solid substrate as described, and then sequentially or concurrently bound to a labeled moiety for detection of the apoptotic bodies or protein, nucleic acid, or phospholipid components thereof

In particular embodiments, the invention provides methods for detecting phospholipid components of apoptotic bodies, the method comprising the step of labeling the phospholipid with a detectably-labeled probe that binds to the phospholipid.

In particular embodiments, the invention provides methods for labeling nucleic acid components of apoptotic bodies, whether that component has been first extracted from the apoptotic body or not, the method comprising the steps of hybridizing to said nucleic acid component of the apoptotic body a primer or probe, preferably a detectably-labeled primer or probe, or a plurality thereof, specific for a nucleic acid species, wherein the nucleic acid is either DNA or RNA or cDNA derived therefrom.

In preferred embodiments, primers or probes are detectably-labeled by conjugation with a radioisotope or a fluorescent moiety or chromophore, thereby permitting the labeled probe to be detected. In a preferred embodiment, the probe is labeled using biotin. The invention provides methods for detecting said labeled-apoptotic probes or moieties bound to the apoptotic body, or to a protein, nucleic acid or phospholipid component thereof, using methods as known in the art, including but not limited to gel electrophoresis, capillary electrophoresis, enzyme-linked immunosorbent assay (ELISA) or modifications thereof, such as using biotinylated or otherwise modified primers, fluorescent-, radioisotope-, or chromogenically-labeled probe, laser-induced fluorescence detection, Western blot analysis, Northern blot analysis, Southern blot analysis, electrochemiluminescence, reverse dot blot detection, high-performance chromatography, spectroscopy including mass spectroscopy and nuclear magnetic resonance spectroscopy, flow cytometry, laser scanning cytometry, or detection at bioelectrical interfaces. Labeled apoptotic bodies can also be detected by flow cytometry using methods in the art, for example, but not a limitation, using methods as described by Baisch et al. (Cell Prolif. 1999, 32: 303-319), incorporated herein by reference in its entirety. Labeled apoptotic bodies can alternatively be detected by laser scanning cytometry using methods in the art, for example, but not a limitation, using methods as described by Bedner et al. (Cytometry 1999, 35: 181-195), incorporated herein by reference in its entirety.

The methods of the invention are advantageously used for providing a diagnosis or prognosis of, or as a predictive indicator for neoplastic and for non-neoplastic diseases or pathologic conditions and injuries. The methods of the invention are particularly useful for providing a diagnosis or prognosis of cancer and premalignancy. The methods of the invention further provide ways to identify animals, most preferably humans, having non-neoplastic disease or pathologic conditions. The invention is particularly advantageous for diagnosis, evaluation, and monitoring of cardiovascular disease such as myocardial disease and of neurologic and neurodegenerative disease such as Alzheimer's disease.

The methods of the invention permit rational, informed treatment options to be used for making therapeutic decisions. The invention may be used either alone, or in conjunction with other diagnostic tests to evaluate disease. The methods of the invention are advantageously use for assessing and monitoring adequacy or efficacy of therapy, or for determining whether additional or more advanced or aggressive therapy is required. The invention therefore provides methods for developing a prognosis in patients. The invention further provides methods for screening individuals to determine a predisposition for a disease or pathologic condition, and further to determine their need for further diagnostic evaluation and/or for preventive therapy. The invention thereby provides methods for evaluating a need for additional testing by radiologic examination, nuclear imaging examination, physical examination, surgery, biopsy, colonoscopy, sigmoidoscopy, bronchoscopy, endoscopy, fine needle aspiration, ductal lavage, stool evaluation, ultrasound, echocardiogram, electrocardiogram, or any other method of medical evaluation.

The invention further provides diagnostic kits useful in the practice of the inventive methods for detecting, diagnosing, monitoring, prognosing or predicting neoplastic or non-neoplastic disease or pathologic condition or injury, wherein the diagnostic kit provides reagents, most preferably primers or probes, and instructions for labeling, isolating, extracting, identifying, amplifying, or detecting apoptotic bodies or their protein, nucleic acid, or phospholipid components, from bodily fluid. In preferred embodiments, the kits further comprise said primers or probes attached to a solid substrate, or a solid particle or bead, more preferably a metallic or magnetic particle or bead.

The methods and reagents of the invention and preferred uses for the methods of the invention are more fully illustrated in the following Examples. These Examples illustrate certain aspects of the above-described method and advantageous results. These Examples are shown by way of illustration and not by way of limitation.

### EXAMPLE 1

A 63 year-old man receiving colon cancer therapy provides by venipuncture a specimen of whole blood into a diagnostic kit wherein a filter separates the cellular component of the blood from the serum fraction containing the apoptotic body fraction. The filtered fraction of the bodily fluid containing apoptotic bodies then is contacted with a solid substrate that specifically binds to the phospholipid component of the apoptotic body. The bound apoptotic body is thereafter labeled with annexin V and detected by immunochemistry. Quantitative evaluation of the presence of apoptotic bodies enables evaluation of therapeutic response to the colon cancer therapy the man is receiving.

### EXAMPLE 2

A 46 year-old woman with a suspicious breast mass provides by venipuncture a blood plasma specimen. The plasma is mixed with magnetic beads coated with capture probes specific to phosphatidylserine on the apoptotic body membrane. Apoptotic bodies present in the plasma are thereby bound to the magnetic beads. The coated magnetic beads are subsequently removed from the plasma under a magnetic field, thereby extracting and concentrating the attached apoptotic bodies. Nucleic acids are then extracted from the extracted apoptotic bodies, and a panel of DNA and RNA species associated with breast cancer, including microsatellite DNA markers, mammoglobulin RNA, and hTERT RNA amplified by polymerase chain reaction and product detected by gel electrophoresis. Detection of these amplified marker fragments informs a diagnosis of breast cancer.

### EXAMPLE 3

A 58 year-old man with a smoking history provides a plasma specimen that is passed through a column of two varying size filters (1.0 micron and 0.1 micron pore size). Particles between 0.1 and 1.0 micron in size are thereby isolated, and further subjected to immunochemistry using an antibody specific to heterogeneous nuclear protein (1 m RNP) A2/B1 protein component. The man's risk for lung cancer is evaluated by detection of apoptotic bodies in his blood plasma.

### EXAMPLE 4

A 42 year-old woman with a pleural effusion of unknown etiology undergoes a diagnostic thoracentesis. An effusion specimen obtained thereby is separated into a non-cellular fraction by filtration through a 0.45 micron filter. The non-cellular fraction is thereafter mixed with a lysis buffer and placed into contact with a bioelectric interface having capture probes that bind telomerase protein or telomerase-associated RNA. A quantitative signal produced thereby enables diagnosis of a malignant effusion.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for extracting, isolating, or concentrating an apoptotic body present in a bodily fluid, the method comprising the steps of:
a) contacting a bodily fluid with a primer or probe specific for a protein, phospholipid, or nucleic acid present in an apoptotic body;
b) conjugating or hybridizing the primer or probe to the protein, phospholipid, or nucleic acid of the apoptotic body; and
c) separating the primer or probe from the bodily fluid, whereby the apoptotic body is thereupon extracted, isolated, or concentrated.

2. The method of claim 1 wherein the primer or probe is an antibody, or oligonucleotide.

3. The method of claim 1 wherein the primer or probe is detectably labeled.

4. The method of claim 1 wherein the bodily fluid is blood, plasma, serum, urine, effusion, ascites, saliva, cerebrospinal fluid, cervical secretions, amniotic fluid, gastrointestinal fluid or secretions, cystic fluid, sputum or bronchial secretions, or breast fluid.

5. A method according to claim 1, comprising the step:
d) extracting nucleic acid from the apoptotic body, and amplifying or signal amplifying said nucleic acid or cDNA derived therefrom in a qualitative or quantitative fashion.

6. The method of claim 3 wherein the labeled probe is labeled annexin V.

7. A method according to claim 1, wherein the primer or probe is attached to a solid substrate.

8. The method of claim 7, wherein the solid substrate is a bead or particle.

9. The method of claim 1, wherein presence of the apoptotic body in the bodily fluid is associated with a cancer or a premalignant condition.

10. The method of claim 1, wherein the bodily fluid is obtained from a human with cancer.

11. The method of claim 1, wherein the bodily fluid is obtained from a human with a non-neoplastic disease.

12. The method of claim 9 further comprising the step of diagnosing, evaluating, or monitoring the cancer or premalignant condition.
